(19) United States
(12) Patent Application Publication
(10) Pub. No.: US 2024/0240240 A1
(43) Pub. Date: Jul. 18, 2024

(54) ENHANCER OLIGONUCLEOTIDES FOR NUCLEIC ACID HYBRIDIZATION

(71) Applicant: Roche Sequencing Solutions, Inc, Pleasanton, CA (US)

(72) Inventors: Rui Chen, Fremont, CA (US); Toumy Guettouche, Pleasanton, CA (US); Donald Sharon, Bristol, NH (US)

(21) Appl. No.: 18/561,986

(22) PCT Filed: May 12, 2022
(86) PCT No.: PCT/EP2022/062890

§ 371 Date: Nov. 17, 2023

Related U.S. Application Data
(60) Provisional application No. 63/192,252, filed on May 24, 2021.

(51) Int. Cl. C12Q 1/6832; C12Q 1/6869; C12Q 1/6886

(52) U.S. Cl. CPC C12Q 1/6832; C12Q 1/6869; C12Q 1/6886; C12Q 2600/106; C12Q 2600/156

(57) ABSTRACT

The invention includes improved methods and compositions for nucleic acid hybridization wherein the improvement comprises the use of enhancer oligonucleotides. Target enrichment is performed using probe oligonucleotides, wherein each probe oligonucleotide comprising a target-binding region, and a first and a second primer-binding region, and one or more enhancer oligonucleotides capable of hybridizing to at least one of the primer binding regions. The forward and reverse primer binding sites can be universal primer binding sites.

### BACKGROUND OF THE INVENTION

Target enrichment (TE) technologies are widely utilized in genomic research including human disease research and clinical applications. These technologies provide focused and cost-efficient solutions as compared with whole-genome analysis such as whole-genome sequencing. By focusing the analysis only on regions of interest in the genome, one can identify disease or phenotype-associated genetic variants and other relevant genomic features, as well as design cost-effective clinical diagnostic assays for such features.

At the outset, target enrichment utilized single-stranded DNA (ssDNA) probes and probe pools for capturing the regions of interest in a high-complexity sample such as a genomic sample. More recently, double-stranded DNA (dsDNA) probes have become popular in TE workflow. DsDNA probes are favored for their ability to capture both the positive (+) and negative (−) strands of the target region, thereby improving data quality by minimizing DNA strand capture bias. Unfortunately, the double-stranded nature of these probes causes self-annealing, cross annealing and other artifacts resulting in decreased assay performance and ultimately, loss of assay sensitivity.

In view of the critical importance of target enrichment in bringing cost-effective genomic analysis to the clinic, there is a need to improve the performance of probes in target enrichment assays.

### SUMMARY OF THE INVENTION

In one embodiment, the invention is a composition for nucleic acid hybridization comprising: two or more probe oligonucleotides, each probe oligonucleotide comprising a target-binding region, and a first and a second primer-binding region, and one or more enhancer oligonucleotides capable of hybridizing to at least one of the primer binding regions. In some embodiments, the two or more probe oligonucleotides comprise a plurality of probe oligonucleotides capable of specifically hybridizing to a plurality of nucleic acid targets under hybridization conditions. In some embodiments, the hybridization conditions are stringent hybridization conditions. In some embodiments, the probe oligonucleotides are double-stranded. In some embodiments, the probe oligonucleotides are single-stranded. In some embodiments, all the probe oligonucleotides have the same first primer-binding region and the same second primer-binding regions. In some embodiments, the enhancer oligonucleotides comprise a mixture of oligonucleotides capable of hybridizing to the first and the second primer-binding regions. In some embodiments, the enhancer oligonucleotides comprise a mixture of oligonucleotides capable of hybridizing to each strand of the first and the second primer-binding regions. In some embodiments, the enhancer oligonucleotides comprise a mixture of four oligonucleotides, each capable of hybridizing to one of the Watson strand or the Crick strand of the first or the second primer-binding regions. In some embodiments, the enhancer oligonucleotides comprise a mixture of more than four oligonucleotides that are grouped into four groups, each group of oligonucleotides capable of hybridizing to one of the Watson strand or the Crick strand of the first or the second primer-binding regions.

In one embodiment, the invention is a composition for nucleic acid target enrichment comprising: two or more probe oligonucleotides, each probe oligonucleotide comprising a target-binding region, and a first and a second primer-binding region, and one or more enhancer oligonucleotides capable of hybridizing to at least one of the primer binding regions. In some embodiments, the two or more probe oligonucleotides comprise a plurality of probe oligonucleotides capable of specifically hybridizing under hybridization conditions, to a plurality of nucleic acid targets present in a mixture with non-target nucleic acids. In some embodiments, the composition further comprises a mixture of target and non-target nucleic acids. In some embodiments,

In one embodiment, the invention is a method of enriching for target nucleic acids, the method comprising: contacting a mixture of target and non-target nucleic acids with a composition comprising two or more probe oligonucleotides, each probe oligonucleotide comprising a target-binding region, and a first and a second primer-binding region, and one or more enhancer oligonucleotides hybridizing to at least one of the primer binding regions; incubating the mixture under hybridization conditions; and separating probe-bound target nucleic acids from unbound nucleic acids. In some embodiments, each of the target nucleic acids, the non-target nucleic acids, the two or more probe oligonucleotides, and the one or more enhancer oligonucleotides is single-stranded. In some embodiments, the method further compresses prior to hybridization, incubating the mixture under conditions that effect denaturation of nucleic acids.

In some embodiments, the mixture of target and non-target nucleic acids constitutes genomic DNA of an organism. In some embodiments, the mixture of target and non-target nucleic acids constitutes a library formed from genomic DNA of an organism. In some embodiments, the library comprises nucleic acids isolated from the organism, each nucleic acid conjugated to at least one adaptor nucleic acid, e.g., two adaptor nucleic acids. In some embodiments, the adaptor nucleic acids include a nucleic acid barcode and universal primer-binding sites.

In some embodiments, the method further comprises removal of any single-stranded nucleic acids from the mixture, e.g., by capturing hybridized nucleic acid via a capture moiety present in the probe oligonucleotides.

In one embodiment, the invention is a method of sequencing nucleic acids comprising: contacting a mixture of target and non-target nucleic acids with a composition comprising two or more probe oligonucleotides, each probe oligonucleotide comprising a target-binding region, and a first and a second primer-binding region, and one or more enhancer oligonucleotides hybridizing to at least one of the primer binding regions; incubating the mixture under hybridization conditions; capturing hybrids formed between the probes and the target nucleic acids to obtain enriched nucleic acids, and sequencing the enriched nucleic acids. In some embodiments, each of the target nucleic acids, the non-target nucleic acids, the two or more probe oligonucleotides, and the one or more enhancer oligonucleotides is single-stranded. In some embodiments, denaturation prior to hybridization is required. In some embodiments, the method further comprises amplifying the enriched nucleic acids, e.g., with universal primers binding to universal primer-binding sites in the enriched nucleic acids. In some embodiments, the invention is an enriched library of nucleic acids formed by a method described herein.

In one embodiment, the invention is a reaction mixture comprising: a plurality of nucleic acids including target and non-target nucleic acids, two or more probe oligonucleotides, each probe oligonucleotide comprising a target-binding region, and a first and a second primer-binding region, one or more enhancer oligonucleotides capable of hybridizing to at least one of the primer binding regions. In some embodiments, the two or more probe oligonucleotides comprise a plurality of probe oligonucleotides capable of specifically hybridizing under hybridization conditions, to a plurality of nucleic acid targets present in a mixture with non-target nucleic acids. In some embodiments, the plurality of nucleic acids including target and non-target nucleic acids constitutes a library formed from genomic DNA of an organism, the library comprising nucleic acids isolated from the organism, each nucleic acid conjugated to at least one adaptor nucleic acid.

In one embodiment, the invention is a method of assessment of a disease or condition in a patient, the method comprising: providing a nucleic acid-containing sample from a patient, enriching target nucleic acids in the sample by the method described herein, determining in the enriched target nucleic acids a mutation status of one or more genetic loci known to be biomarkers of the disease or condition, thereby detecting the disease or condition in the patient.

In one embodiment, the invention is a method of selecting a treatment a disease or condition in a patient, the method comprising: providing a nucleic acid-containing sample from a patient having a disease or condition, enriching target nucleic acids in the sample by the method described herein, determining in the enriched target nucleic acids a mutation status of one or more genetic loci known to be biomarkers of the disease or condition, and selecting a treatment appropriate for the mutations detected in the enriched nucleic acids.

In one embodiment, the invention is a method of diagnosing or screening for the presence of a cancerous tumor in a patient, the method comprising: providing a nucleic acid-containing sample from a patient, enriching target nucleic acids in the sample by the method described herein, determining in the enriched nucleic acids a mutation status of one or more genetic loci known to indicate the presence of a cancerous tumor, thereby detecting the presence of the cancerous tumor in the patient.

In one embodiment, the invention is a method of selecting a treatment targeting the cancerous tumor in a patient based on the mutation status of the tumor, the method comprising: providing a nucleic acid-containing sample from a patient, enriching target nucleic acids in the sample by the method described herein, determining in the enriched nucleic acids a mutation status of one or more genetic loci known to be mutated a cancerous tumor, and selecting a treatment targeting the mutant status found.

In one embodiment, the invention is a method of monitoring the growth or shrinkage of a tumor, the method comprising: periodically sampling circulating cell-free DNA (cfDNA) from a patient, enriching for one or more target sequences in the cfDNA by the method described herein, detecting changes in the amount mutated cfDNA containing one or more mutations in the target sequences known to mutated in a cancerous tumor, wherein an increase in the level of such mutated cfDNA indicates tumor growth, while a decrease in the level of such mutated cfDNA indicates tumor shrinkage.

In one embodiment, the invention is a method of monitoring the effectiveness of treatment of cancer in a patient, the method comprising: periodically sampling circulating cell-free DNA (cfDNA) from a patient, enriching for one or more target sequences in the cfDNA by the method described herein, detecting changes in the amount cfDNA containing one or more mutations in the target sequences known to mutated in a cancerous tumor, wherein an increase in the level of such mutant cf DNA indicates tumor growth and ineffectiveness of treatment, while a decrease in the level of such mutant cfDNA indicates tumor shrinkage and effectiveness of treatment, and a stable level of such mutant cfDNA indicates stable disease and effectiveness of treatment.

In one embodiment, the invention is a method of diagnosis or minimal residual disease (MRD) in a cancer patient, the method comprising: obtaining circulating cell-free DNA (cfDNA) from a patient, enriching for one or more target sequences in the cfDNA by the method described herein, detecting in the enriched cfDNA a mutation status of one or more genetic loci known to mutated in a cancerous tumor, wherein the presence of the mutated cfDNA indicates the presence of MRD in the patient.

In one embodiment, the invention is a kit for improved hybridization of nucleic acids comprising: one or more probe oligonucleotides, each probe oligonucleotide comprising a target-binding region, and a first and a second primer-binding region, one or more enhancer oligonucleotides capable of hybridizing to at least one of the primer binding regions. In some embodiments, the one or more probe oligonucleotides are double-stranded and the kit includes four enhancer oligonucleotides capable of hybridizing to four primer binding regions. In some embodiments, the kit comprises one or more of the following: reagents for purification and separation of nucleic acids, reagents for forming a library of nucleic acids, reagents for amplifying nucleic acids and reagents for sequencing nucleic acids.

In one embodiment, the invention is a method of enriching for target nucleic acids, the method comprising: contacting a mixture of target and non-target nucleic acids with a composition comprising: two or more probe oligonucleotides, each probe oligonucleotide comprising a target-binding region, and a first and a second primer-binding region, wherein the first primer binding region is hybridized to a capture oligonucleotide attached to a solid support; one or more enhancer oligonucleotides hybridizing to the second primer binding region; incubating the mixture under hybridization conditions; contacting the mixture with one or more enhancer oligonucleotides hybridizing to the first primer binding region under conditions suitable for dissociation of the first primer binding region from the capture oligonucleotide thereby separating probe-bound target nucleic acids from unbound nucleic acids.

### BRIEF DESCRIPTION OF THE DRAWINGS

FIGS. 1A, 1B and 1C are diagrams illustrating the design and operation of the enhancer oligonucleotides.

FIGS. 2A and 2B show the results of sequencing performed on nucleic acids enriched by hybridization in the presence of the enhancer oligonucleotides

### DETAILED DESCRIPTION OF THE INVENTION

### Definitions

The following definitions aid in understanding the disclosure. All terms of art not specifically defined in this section have the ordinary and customary meaning.

The term “probe” refers to a nucleic acid (either single stranded or double-stranded), including an oligonucleotide that is capable of specifically binding to a target nucleic acid under stringent hybridization conditions.

The term “oligonucleotide” refers to a nucleic acid that is typically shorter than a natural occurring nucleic acid. The terms oligonucleotide and nucleic acid may be used interchangeably. Unless stated otherwise, an oligonucleotide is single stranded.

The term “enhancer oligonucleotide” refers to the type of oligonucleotide described and claimed herein that has a specific property of hybridizing to certain elements present in hybridization probes that improving the performance of the hybridization probes.

The term “blocker oligonucleotide” refers to an oligonucleotides that is added to a hybridization reaction involving nucleic acid libraries prepared, e.g., for sequencing. The blocker oligonucleotide has a specific property of hybridizing to and blocking certain elements present in all library molecules. Some commercially available blocker oligonucleotides are sold under a name “universal enhancer oligonucleotides.” For the avoidance of doubt, the terms “enhancer oligonucleotide” as defined herein is distinct from “universal enhancer oligonucleotide.” The term “universal enhancer oligonucleotide” is not used in this disclosure.

The term “primer binding region” includes a primer binding site which is a sequence within the nucleic acid where an amplification primer binds to initiate strand synthesis. In the context of this disclosure, the term “primer binding region” further includes a reverse complement of the primer binding site. For example, a double stranded nucleic acid resulting from amplification with primers includes four primer binding regions, one region at each of the two ends of each of the two strands, wherein two of the primer binding regions are primer binding sites and the other two of the primer binding regions are reverse complements of the primer binding sites.

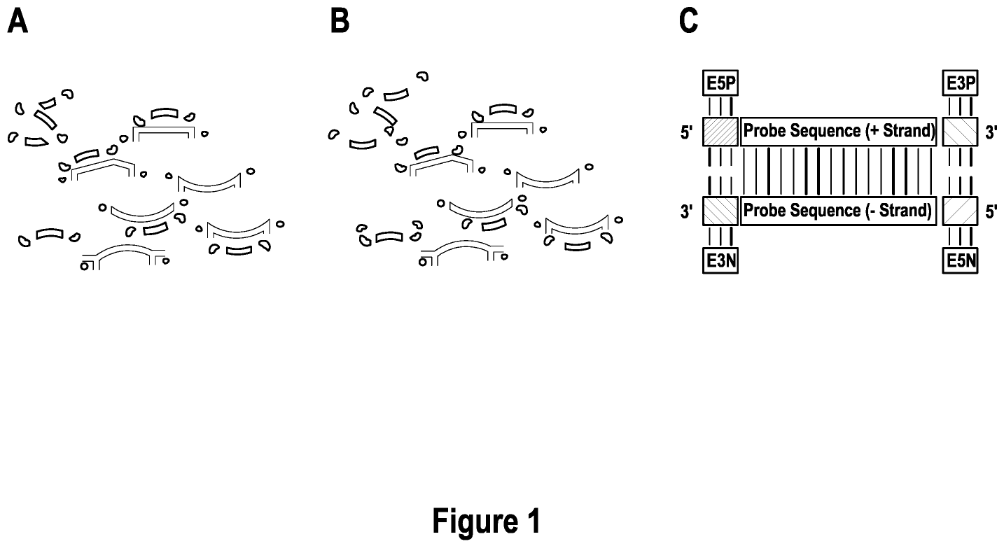

Target enrichment (TE) technologies are widely utilized in genomic research as part of life sciences and human disease research and clinical applications. Target enrichment provides focused and cost-efficient solutions as compared to whole genome sequencing in the identification of disease and phenotype-associated genetic variants and genomic regions. Double-stranded DNA (dsDNA) probes have become a popular type of probes in TE workflows in recent years, for their ability to capture both the positive (+) and negative (−) strands of a target region to be enriched. The dsDNA probes improve data quality by minimizing DNA strand capture bias. To control production cost, the leading dsDNA probe providers manufacture large quantities of these probes through amplification by polymerase chain reaction (PCR). To enable PCR, one must include primer-binding sites (PBS) at the ends of each dsDNA probe being produced. PBS are usually identical on all probes synthesized by a manufacturer as part of a lot or pool of probes. While reducing manufacturing costs, these production primer-binding sites lead to the formation of artifacts that impair probe performance. The reduction in performance is due to tendency of a positive (+) strand and a negative (−) strand of the probe molecules to concatenate (FIG. 1A, top left) or to self-anneal or cross-anneal (FIG. 1A, bottom left) through these complementary PBS. These artifacts negatively affect hybridization efficiency, thus leading to suboptimal target enrichment and lower quality of the downstream analysis, such as for example, nucleic acid sequencing.

Hybridization blockers are known in the art. However, hybridization blocker oligonucleotides are traditionally used to block adaptor sequences in the library of nucleic acids, see e.g., US20200102611. During target enrichment hybridizations, such blocker oligonucleotides bind to library molecules and not to the hybridization probes. The existing blocker oligonucleotides prevent adaptor-adaptor hybridization of the library molecules and do not address any of the problems or artefacts related to hybridization probes. For example, the problems of concatenation, cross-annealing or self-annealing of hybridization probes are not addressed by the existing blockers.

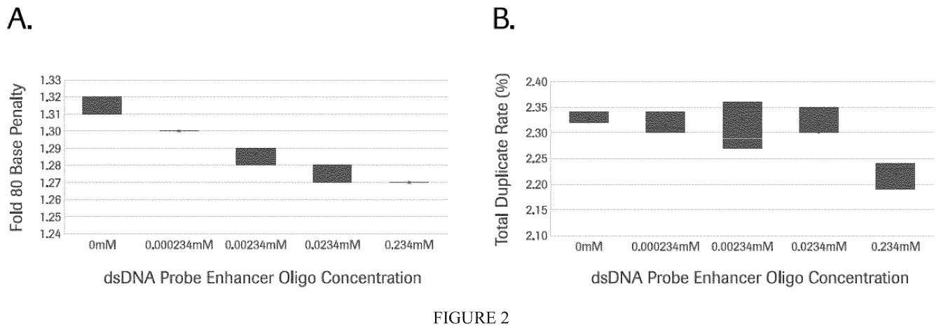

The instant disclosure provides a solution to problems related to hybridization probes, e.g., target enrichment hybridization probes. The instant invention comprises Probe Enhancer Oligonucleotides (dPEOs) that improve capture efficiency and target enrichment performance. The enhancer oligonucleotides are designed to bind to common sequences shared among the pool of hybridization probes. In some embodiments, enhancer oligonucleotides are designed to bind to primer binding sites present in dsDNA probes. PCR is commonly employed in the manufacture of hybridization probes. In such instances, each probe contains a forward and a reverse universal primer binding sites. The enhancer oligonucleotides of the instant invention are designed to bind these universal sites and prevent any undesirable interactions between the probes in the hybridization mixture. As a result, the enhancer oligonucleotides minimize probe concatenation (illustrated in FIG. 1B, top left) and reduce the prevalence of re-annealed or cross-annealed double-stranded probes (FIG. 1B, bottom left), thus increasing the number of effective probe molecules in hybridization reactions. As shown in FIGS. 2A and 2B, review of the sequencing data generated with different doses of enhancer oligonucleotides, the use of enhancer oligonucleotides improves capture uniformity (FIG. 2A) and decreases read duplicate levels (FIG. 2B), in a dose dependent manner.

The various aspects of the invention are described in further detail below.

The present invention involves a method of manipulating nucleic acids from a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids that may contain nucleic acids (e.g., urine, sputum, serum, blood or blood fractions, i.e., plasma, lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). In some embodiments, the sample is a blood plasma sample or a urine sample containing cell-free DNA (cfDNA), including circulating tumor DNA (ctDNA). In other embodiments, the sample is a cultured sample, e.g., a tissue culture containing cells and fluids from which nucleic acids may be isolated. In some embodiments, the nucleic acids of interest in the sample come from infectious agents such as viruses, bacteria, protozoa or fungi.

The present invention involves manipulating isolated nucleic acids isolated or extracted from a sample. Methods of nucleic acid extraction are well known in the art. See J. Sambrook et al., “Molecular Cloning: A Laboratory Manual,” 1989, 2nd Ed., Cold Spring Harbor Laboratory Press: New York, N.Y.). A variety of kits are commercially available for extracting nucleic acids (DNA or RNA) from biological samples (e.g., KAPA Express Extract (Roche Sequencing Solutions, Pleasanton, Cal.) and other similar products from BD Biosciences Clontech (Palo Alto, Cal.), Epicentre Technologies (Madison, Wisc.); Gentra Systems, (Minneapolis, Minn.); and Qiagen (Valencia, Cal.), Ambion (Austin, Tex.); BioRad Laboratories (Hercules, Cal.); and more.

In some embodiments, nucleic acids are extracted, separated by size and optionally, concentrated by epitachophoresis as described e.g., in publications WO2019092269 and WO2020074742.

Target enrichment is a method of capturing one or more target nucleic acids or separating the one or more target nucleic acid from any non-target nucleic acids in a sample or reaction mixture. In some embodiments, target enrichment is a method of increasing the concentration of one or more target nucleic acids relative to the concentration of any non-target nucleic acids present in a sample or reaction mixture.

Target nucleic acids are the nucleic acid of interest that may be present in the sample. Each target is characterized by its nucleic acid sequence. In some embodiments, the target nucleic acid is a gene or a gene fragment (including exons and introns). In some embodiments, the target is a gene, gene fragment or inter-genic region involved in a fusion event, e.g., a region where a fusion breakpoint is located. In some embodiments, the target is present in RNA and is a gene transcript or a portion thereof. In some embodiments, the target nucleic acid comprises a biomarker, i.e., a gene whose variants such as single nucleotide variation (SNV), copy number variation (CNV) or gene fusion are associated with a disease or condition. For example, the target nucleic acids can be selected from panels of disease-relevant markers described in U.S. patent application Ser. No. 14/774,518 filed on Sep. 10, 2015. Such panels are available as AVENIO ctDNA Analysis kits (Roche Sequencing Solutions, Pleasanton, Cal.). In some embodiments, the target nucleic acids are one or more of the genes listed in Table 1 or Table 2.

TABLE 1
Composition of the expanded biomarker panel

| | | | | | |
|---|---|---|---|---|---|
| APC | KRAS | ABL1 | FGFR3 | JAK3 | RAF1 |
| BRCA1 | MET | AKT1 | FLT1 | KDR | RNF43 |
| BRCA2 | TP53 | AKT2 | FLT3 | MAP2K1 | TERT |
| | | | | | promoter |
| EGFR | KIT | ARAF | FLT4 | MAP2K2 | TSC1 |
| ERBB2 | NRAS | CDK6 | GATA3 | MTOR | TSC2 |
| ALK | PDGFRA | CSF1R | GNA11 | NFE2L2 | PTEN |
| BRAF | RET | CTNNB1 | GNAQ | NTRK1 | RB1 |
| DPYD | ROS1 | DDR2 | GNAS | PDGFRB | SMAD4 |
| AR | MSH2 | EZH2 | IDH1 | PIK3CA | SMO |
| CCND1 | MSH6 | FGFR1 | IDH2 | PIK3R1 | STK11 |
| CCND2 | NF2 | FGFR2 | JAK2 | PTCH1 | VHL |
| CCND3 | PDCD1LG2 | CDK4 | ESR1 | KEAP1 | UGT1A1 |
| CD274 | PMS2 | CDKN2A | FBXW7 | MLH1 | |

TABLE 2
Composition of the surveillance biomarker panel

| | | | | | | |
|---|---|---|---|---|---|---|
| ABCC5 | CSMD1 | FAT1 | HTR1E | MAP7D3 | PIK3CA | SV2A |
| ABCG2 | CSMD3 | FBN2 | HTR2C | MKRN3 | PIK3CG | T |
| ACTN2 | CTNNB1 | FBXL7 | IFI16 | MMP16 | PKHD1L1 | THSD7A |
| ADAMTS12 | CTNND2 | FBXW7 | IL7R | MTX1 | POLE | TIAM1 |
| ADAMTS16 | CYBB | FCRL5 | INSL3 | MYH7 | POM121L12 | TMEM200A |
| ARFGEF1 | DCAF12L1 | FOXG1 | ITGA10 | MYT1L | PREX1 | TNFRSF21 |
| ASTN1 | DCAF12L2 | FRYL | ITSN1 | NAV3 | PTPLA | TNN |
| ASTN2 | DCAF4L2 | GBA3 | KCNA5 | NEUROD4 | RALYL | TNR |
| AVPR1A | DCLK1 | GBP7 | KCNB2 | NFE2L2 | RFX5 | TRHDE |
| BCHE | DCSTAMP | GJA8 | KCNC2 | NLGN4X | RIN3 | TRIM58 |
| BPIFB4 | DDI1 | GPR139 | KCNJ3 | NLRP3 | RNASE3 | TRPS1 |
| C6 | DLGAP2 | GRIA2 | KCTD8 | NMUR1 | ROBO2 | UGT3A2 |
| C6orf118 | DMD | GRIK3 | KEAP1 | NOL4 | SEMA5B | USH2A |
| CA10 | DNTTIP1 | GRIN2B | KIAA1211 | NPAP1 | SLC18A3 | USP29 |
| CACNA1E | DOCK3 | GRIN3B | KIF17 | NR0B1 | SLC39A12 | VPS13B |
| CDH12 | DSC3 | GRM1 | KIF19 | NRXN1 | SLC6A5 | WBSCR17 |
| CDH18 | DSCAM | GRM5 | KLHL31 | NXPH4 | SLC8A1 | WIPF1 |
| CDH8 | EGFLAM | GRM8 | KPRP | NYAP2 | SLITRK1 | WSCD2 |
| CDH9 | EPHA5 | GSX1 | LPPR4 | OPRD1 | SLITRK4 | ZC3H12A |
| CDKN2A | EPHA6 | HCN1 | LRFN5 | P2RY10 | SLITRK5 | ZFPM2 |
| CHRM2 | EYS | HCRTR2 | LRP1B | PAX6 | SLPI | ZIC1 |
| CNTN5 | FAM135B | HEBP1 | LRRC7 | PCDH15 | SMAD4 | ZIC4 |
| CNTNAP2 | FAM151A | HECW1 | LRRTM1 | PDYN | SOX9 | ZNF521 |
| CPXCR1 | FAM5B | HS3ST4 | LRRTM4 | PDZRN3 | SPTA1 | ZSCAN1 |
| CPZ | FAM5C | HS3ST5 | LTBP4 | PGK2 | ST6GALNAC3 | KIT |
| CRMP1 | FAM71B | HTR1A | MAP2 | PHACTR1 | STK11 | NRAS |
| APC | KRAS | ALK | PDGFRA | MET | BRAF | RET |
| BRCA1 | BRCA2 | TP53 | DPYD | EGFR | ERBB2 | UGT1A1 |

In some embodiments, the target nucleic acids are one or more genes involved in clinically-relevant gene fusions. In some embodiments, the target nucleic acids are one or more genes known to undergo fusions in tumors. In some embodiments, the target nucleic acids are one or more fusion sites associated with the genes ALK, RET, ROS, FGFR2, FGFR3, NTRK1, ALK, PPARG, BRAF, EGFR, FGFR1, FGFR2, FGFR3, MET, NRG1, NTRK1, NTRK2, NTRK3, RET, ROS1, AXL, PDGFRA, PDGFB, ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAX5, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In some embodiments, the target nucleic acids are one or more genes or genomic regions involved in epigenetic modifications, such as DNA methylation. In some embodiments, the target nucleic acids are one or more genes involve in genome maintenance or mismatch repair. In some embodiments, the target nucleic acids include microsatellite loci exhibiting microsatellite instability (MSI). In some embodiments, the target nucleic acids include one or more genes involved in mismatch repair which when mutated, are known to confer a microsatellite instability (MSI) phenotype.

In some embodiments, the target nucleic acid is RNA (including mRNA). In some embodiments, the target nucleic acid is cDNA derived from RNA e.g., via reverse transcription. In some embodiments, the target nucleic acid is DNA, including cellular DNA or cell-free DNA (cfDNA) including circulating tumor DNA (ctDNA) and cell-free fetal DNA. The target nucleic acid may be present in a short or long form. In some embodiments, longer target nucleic acids are fragmented by enzymatic or physical treatment as described below. In some embodiments, the target nucleic acid is naturally fragmented, e.g., includes circulating cell-free DNA (cfDNA) or chemically degraded DNA such as the one found in chemically preserved or ancient samples.

The instant invention involves the use of hybridization probes targeting the nucleic acids of interest in a sample (target nucleic acids). Hybridization probes are either single-stranded or double-stranded nucleic acids. In some embodiments, the probes are pool of more than one, e.g., up to 10, or 10-100 probes, or 100-500 probes, or 500-1,000, or 1,000-10,000 probes. In some embodiments, one probe is present for each target locus, i.e., a gene or a region of interest. In other embodiments, multiple probes, e.g., 2-10, or 10-100 probes, or 100-500 probes are present covering the same gene or region of interest. Many organism-specific hybridization probes and probe pools, including custom-made probes and probe pools are available. Commonly, hybridization probes are manufactured via a workflow that includes amplification e.g., by PCR or a non-exponential amplification method. For this reason, the probes contain amplification primer binding sites such as e.g., universal primer binding sites.

The instant invention involves the use of enhancer oligonucleotides specific for amplification primer binding sites such as e.g., universal primer binding sites in the probes. These enhancer oligonucleotides are distinct from “universal enhancer oligonucleotides” currently available (e.g., as part of the KAPA HyperCap workflow). The existing universal enhancer oligonucleotides bind adaptor sequences in the library molecules. By contrast, the enhancer oligonucleotides of the instant invention are designed to bind primer binding sites in the hybridization probes. (FIG. 1B). In some embodiments, four enhancer oligonucleotides are added, each complementary to the forward and reverse primer binding sites, and reverse complementary to the forward and reverse primer binding sites in double stranded probe oligonucleotides as shown in FIG. 1B. In other embodiments, e.g., where probes are single stranded, fewer than four enhancer oligonucleotides described above are added.

In some embodiments, the enhancer oligonucleotides have the same length as the primer binding sites. In other embodiments, the enhancer oligonucleotides are shorter or longer than the primer binding sites. One of skill in art is able to determine an optimal length of an enhancer oligonucleotide so that at given hybridization conditions (e.g., the conditions used in target enrichment), the enhancer oligonucleotides form stable hybrids with the primer binding sites in the hybridization probes thus achieving the desired hybridization enhancement described herein.

One of skill in the art is further able to calculate a desired ratio between the enhancer oligonucleotides and hybridization probes in view of the fact that depending on the number of enhancer oligonucleotides used, between one and four enhancer oligonucleotides are needed to bind each double-stranded hybridization probe. In some embodiments, the molar ratio of probes to enhancer oligonucleotides is 1:4. In other embodiments, molar excess of enhancer oligonucleotides is added so that the molar ratio of probes to enhancer oligonucleotides is 1:6, 1:8, 1:10 or higher. In some embodiments, the final concentration of enhancer oligonucleotides is about 0.2 mM, 0.02 mM, 0.002 mM, or 0.0002 mM. As a general rule, it may be beneficial to have a molar excess of the enhancer oligonucleotide to the probes.

It may be beneficial to optimize the design of the enhancer oligonucleotides to have the desired melting by temperature (Tₘ) under the hybridization conditions employed in the target enrichment process. In some embodiments, the predicted Tₘ of an enhancer oligonucleotide is determined experimentally or using a manual calculation or any of the in silico tools available for this purpose. In some embodiments, the desired Tₘ of enhancer oligonucleotides is higher than the incubation temperature used in the hybridization conditions employed in the target enrichment. In some embodiments, the desired Tₘ of enhancer oligonucleotides is higher than the Tₘ of a hypothetical probe-probe hybrid or higher than the Tₘ of a double-stranded probe. To achieve such a high Tₘ, in some embodiments, the enhancer oligonucleotides comprise one or more modified nucleotides or nucleotide modifications selected from: e.g., 5-methyl cytosine, 2,6-diaminopurine, 5-hydroxybutynl-2′-deoxyuridine, 8-aza-7-deazaguanosine, a ribonucleotide, a 2′O-methyl ribonucleotide or a locked nucleic acid.

The length of the enhancer oligonucleotide also influences the melting temperature. The primer binding sites are more often about 10-20 nucleotides long but may be between about 10 and about 40 nucleotides long. It is not necessary that the length of the enhancer oligonucleotide exactly match the length of the primer binding site to be blocked. For example, the enhancer oligonucleotide may be one or more nucleotides shorter than the primer binding site to be blocked on one or both sides of the enhancer oligonucleotide.

It is also not necessary that the enchanter oligonucleotide be perfectly complementary to the primer binding site to be blocked. In some embodiments, the enhancer oligonucleotide is less than 100% complementary, e.g., >90%, 80-90%, or 70-80% complementary to the primer binding site to be blocked.

In some embodiments, the nucleic acids in the sample are present in the form of a library. In some embodiments, the library is formed from genomic DNA of an organism. In such embodiments, the library is a genomic library. The library consists of a plurality of nucleic acids modified to enable a downstream application such as sequencing, amplification or another type of detection method. A library is formed from a plurality of nucleic acids in a sample e.g., by adding one or more common elements to the plurality of nucleic acids in the sample.

In some embodiments, the library if formed by adding common adaptor molecules to one or both ends of the nucleic acids in the sample. Adaptors of various shapes and functions are known in the art (see e.g., PCT/EP2019/05515 filed on Feb. 28, 2019, U.S. Pat. Nos. 8,822,150 and 8,455,193). In some embodiments, the adaptor comprises certain elements such as nucleic acid barcodes, primer binding sites and ligation-enabling site. The adaptor includes at least one element selected from the following: a barcode, a primer binding site, and a ligation-enabling site. The adaptor may be double-stranded, partially single stranded or single stranded. In some embodiments, a Y-shaped, a hairpin adaptor or a stem-loop adaptor is used wherein the double-stranded portion of the adaptor is ligated to the double stranded nucleic acid formed as described herein. In some embodiments, adaptors are in vitro synthesized artificial sequences. In other embodiments, adaptors are in vitro synthesized naturally-occurring sequences. In yet other embodiments, adaptors are isolated naturally occurring molecules or isolated non naturally-occurring molecules.

In some embodiments, adaptors are added by extending an adaptor sequence-containing primer annealed to the plurality of nucleic acids in the sample. Such primer are referred to as “tailed primers.” A tailed primer comprises a target-hybridizing 3′-portion and a non-hybridizing 5′-tail containing the adaptor sequence. In some embodiments, the target-hybridizing sequence is specific to one nucleic acid in the library, e.g., gene-specific. In some embodiments, the target-hybridizing sequence is specific to one type of nucleic acids, e.g., a poly-T sequence. In some embodiments, the target-hybridizing sequence is random, e.g., a random hexamer nucleotide sequence. Upon extension of tailed primers hybridized to the nucleic acids in a sample, the nucleic acids form a library of adapted nucleic acids.

In some embodiments, adaptors are added by ligation to the ends of each of plurality of nucleic acids in a sample. In some embodiment, adaptors are double-stranded or partially double-stranded adaptor oligonucleotides with overhangs or with blunt ends. In some embodiments, the double-stranded DNA may comprise blunt ends to which a blunt-end ligation can be applied to ligate a blunt-ended adaptor. In other embodiments, the blunt ended DNA undergoes A-tailing where a single A nucleotide is added to the 3′-end of the blunt ends. A corresponding adaptor is designed to have a single T nucleotide extending from the 3′-end of a blunt end to facilitate ligation between the DNA and the adaptor. Commercially available kits for performing adaptor ligation include AVENIO ctDNA Library Prep Kit or KAPA HyperPrep and HyperPlus kits (Roche Sequencing Solutions, Pleasanton, CA). In some embodiments, the adaptor-ligated (adapted) library nucleic acids may be separated from excess adaptors and unligated nucleic acids in the sample.

In some embodiments, adaptors present in the library nucleic acids are used in sequencing the nucleic acids. Analyzing individual molecules by massively parallel sequencing typically requires a separate level of barcoding for sample identification and error correction. The use of molecular barcodes such as described in U.S. Pat. Nos. 7,393,665, 8,168,385, 8,481,292, 8,685,678, and 8,722,368. A unique molecular barcode is added to each molecule to be sequenced to mark molecule and its progeny (e.g., the original molecule and its amplicons generated by PCR). The unique molecular identifier barcode (UID) (also known as unique molecular identifier (UMI)) has multiple uses including counting the number of original target molecules in the sample and error correction (Newman, A., et al., (2014) An ultrasensitive method for quantitating circulating tumor DNA with broad patient coverage, Nature Medicine doi:10.1038/nm.3519).

In some embodiments, unique molecular barcodes (UIDs) are used for sequencing error correction. The entire progeny of a single target molecule is marked with the same barcode and forms a barcoded family. A variation in the sequence not shared by all members of the barcoded family is discarded as an artefact. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments of the invention, the adaptor ligated to one or both ends of the barcoded target nucleic acid comprises one or more barcodes used in sequencing. A barcode can be a UID or a multiplex sample ID (MID or SID) used to identify the source of the sample where samples are mixed (multiplexed). The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the invention, the barcodes are between about 4-20 bases long so that between 96 and 384 different adaptors, each with a different pair of identical barcodes are added to a human genomic sample. In some embodiments, the number of UIDs in the reaction can be in excess of the number of molecules to be labelled. A person of ordinary skill would recognize that the number of barcodes depends on the complexity of the sample (i.e., expected number of unique target molecules) and would be able to create a suitable number of barcodes for each experiment.

In some embodiments, the invention is an improved method of enriching for one or more target nucleic acids present in a sample or reaction mixture also comprising non-target nucleic acids. The invention comprises contacting the sample or reaction mixture with one or more probes that specifically hybridize to the target nucleic acids. More specifically, the invention comprises the use of an improved probe mixture. The improved probe mixture comprises two or more probe oligonucleotides, e.g., a plurality of probe oligonucleotides. In some embodiments, the plurality of probe comprises fewer 2, 3, 4, 5, 6, 7, 8, 9, 10 or 10-100 probes, or 100-500 probes, or 500-1,000, or 1,000-10,000 probes. One or more of the probes in the probe mixture include amplification primer binding regions. The improved probe mixture further comprises hybridization enhancer oligonucleotides capable of hybridizing to the primer binding regions in the probes. In some embodiments, the probe mixture that contains one or more enhancer oligonucleotides hybridizing to at least one of the primer binding regions. In some embodiments, the probe mixture comprises enhancer oligonucleotides capable of hybridizing to the first and the second primer-binding regions in the probes. In some embodiments, the molar ratio of the probes to the enhancer oligonucleotides in the probe mixture is optimized to achieve blocking without cross-reaction of probes with additional hybridization sites, such as partially complementary sites. In some embodiments, the molar ration of probe oligonucleotides to the enhancer oligonucleotides is 1:2, 1:4, 1:8 or higher.

The method further comprises incubating the reaction mixture comprising the target nucleic acids, the non-target nucleic acids, the probes and the enhancer oligonucleotides under hybridization conditions and separating the target nucleic acids hybridized to the probed from non-hybridized nucleic acids.

In some embodiments, the nucleic acids in the mixture including the target nucleic acids, the non-target nucleic acids, the two or more probe oligonucleotides, and the one or more enhancer oligonucleotides are single-stranded. In some embodiments, at least one of the nucleic acids in the mixture including the target nucleic acids, the non-target nucleic acids, the two or more probe oligonucleotides, and the one or more enhancer oligonucleotides is double-stranded and the method includes a preliminary step of incubating the sample or reaction mixture under conditions that effect denaturation of nucleic acids. Denaturation of nucleic acids may be effected by elevated temperature, alkali or a combination thereof.

In some embodiments, the target enrichment procedure described herein is performed on a genomic DNA of an organism. In some embodiments, genomic DNA of an organism is converted into a genomic library prior to the target enrichment procedure described herein. In some embodiments, the genomic DNA or the genomic DNA library is depleted of repetitive sequences prior to the target enrichment procedure described herein.

In some embodiments, depletion of the repetitive sequences from the genomic DNA or the genomic DNA library is performed by the target enrichment method described herein, i.e., the hybridization procedure utilizing the improved probe mixture described herein, is applied to the probes capable of hybridizing to repeated sequences in the genome of the organism.

In some embodiments, the method further comprises after hybridization, removal of any unhybridized nucleic acids or any single-stranded nucleic acids from the reaction mixture. In some embodiments, the unhybridized or single-stranded nucleic acids are removed by capture. In some embodiments, the hybridization probes comprise a capture moiety (e.g., biotin) enabling capture of sample nucleic acids hybridized to the probes.

In some embodiments, the invention is an economical method of sequencing nucleic acids comprising contacting a mixture of target and non-target nucleic acids with a composition comprising two or more probe oligonucleotides, each probe oligonucleotide comprising a target-binding region, and a first and a second primer-binding region, and one or more enhancer oligonucleotides hybridizing to at least one of the primer binding regions; incubating the mixture under hybridization conditions; capturing the hybridized target nucleic acids and sequencing only the captured nucleic acids. In some embodiments, the economical sequencing method is applied to a genomic DNA of an organism. In some embodiments, genomic DNA of an organism is converted into a genomic library prior to the sequencing procedure.

In some embodiments, the method further comprises amplifying the enriched nucleic acids prior to sequencing. In some embodiments, amplification prior to sequencing utilizes universal primer-binding cites present in the adaptors of the library nucleic acids.

In some embodiments, the invention includes a step of amplifying the nucleic acids. In some embodiments, amplification occurs prior to the sequencing step. In some embodiments, amplification occurs prior to the target enrichment step. In some embodiments, amplification occurs after the target enrichment step but prior to the sequencing step. The amplification utilizes an upstream primer and a downstream primer. In some embodiments, both primers are target specific primers, i.e., primers comprising a sequence complementary to the target sequence of the methylation biomarker. In other embodiments, one or both primers are universal primers. In some embodiments, universal primer binding sites are present in adaptors ligated to the target sequenced as described herein. In some embodiments, a universal primer binding site is present in the 5′-region (tail) of a target-specific primer. Accordingly, after one or more rounds of primer extension with a tailed target-specific primer, a universal primer may be used for subsequent rounds of amplification. In some embodiments, a universal primer in paired with another universal primer (of the same or different sequence). In other embodiments, a universal primer is paired with a target-specific primer.

In some embodiments, the nucleic acids enriched by the method described herein are sequenced. Any of a number of sequencing technologies or sequencing assays can be utilized. The term “Next Generation Sequencing (NGS)” as used herein refers to sequencing methods that allow for massively parallel sequencing of clonally amplified molecules and of single nucleic acid molecules.

Non-limiting examples of sequence assays that are suitable for use with the methods disclosed herein include nanopore sequencing (U.S. Pat. Publ. Nos. 2013/0244340, 2013/0264207, 2014/0134616, 2015/0119259 and 2015/0337366), Sanger sequencing, capillary array sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), sequencing by hybridization (Drmanac et al., Nature Biotech., 16:54-58 (1998), and NGS methods, including but not limited to sequencing by synthesis (e.g., HiSeq™, MiSeq™, or Genome Analyzer, each available from Illumina), sequencing by ligation (e.g., SOLID™, Life Technologies), ion semiconductor sequencing (e.g., Ion Torrent™, Life Technologies), and SMRT® sequencing (e.g., Pacific Biosciences).

Commercially available sequencing technologies include: sequencing-by-hybridization platforms from Affymetrix Inc. (Sunnyvale, Calif.), sequencing-by-synthesis platforms from Illumina/Solexa (San Diego, Calif.) and Helicos Biosciences (Cambridge, Mass.), sequencing-by-ligation platform from Applied Biosystems (Foster City, Calif.). Other sequencing technologies include, but are not limited to, the Ion Torrent technology (ThermoFisher Scientific), and nanopore sequencing (Genia Technology from Roche Sequencing Solutions, Santa Clara, Cal.), and Oxford Nanopore Technologies (Oxford, UK).

In some embodiments, the sequencing step involves sequence aligning. In some embodiments, aligning is used to determine a consensus sequence from a plurality of sequences, e.g., a plurality having the same unique molecular ID (UID). The molecular ID is a barcode that can be added to each molecule prior to sequencing or if amplification step is included, prior to the amplification step. In some embodiments, a UID is present in the 5′-portion of the RT primer. Similarly, a UID can be present in the 5′-end of the last barcode subunit to be added to the compound barcode. In other embodiments, a UID is present in an adaptor and is added to one or both ends of the target nucleic acid by ligation.

In some embodiments, a consensus sequence is determined from a plurality of sequences all having an identical UID. The sequenced having an identical UID are presumed to derive from the same original molecule through amplification. In other embodiments, UID is used to eliminate artifacts, i.e., variations existing in the progeny of a single molecule (characterized by a particular UID). Such artifacts resulting from PCR errors or sequencing errors can be eliminated using UIDs.

In some embodiments, the number of each sequence in the sample can be quantified by quantifying relative numbers of sequences with each UID among the population having the same multiplex sample ID (MID). Each UID represents a single molecule in the original sample and counting different UIDs associated with each sequence variant can determine the fraction of each sequence variant in the original sample, where all molecules share the same MID. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

In some embodiments, the invention is a composition for nucleic acid hybridization comprising: two or more probe oligonucleotides, each probe oligonucleotide comprising a target-binding region, and a first and a second primer-binding region, and one or more enhancer oligonucleotides capable of hybridizing to at least one of the primer binding regions. In some embodiments, the composition results from contacting a sample with probe mixture comprising a plurality of probe oligonucleotides capable of specifically hybridizing to a plurality of nucleic acid targets under hybridization conditions. The probe mixture further comprises enhancer oligonucleotides comprising a mixture of oligonucleotides capable of hybridizing to the first and the second primer-binding regions. Various mixtures of enhancer oligonucleotides are envisioned in the scope of this invention. For example, oligonucleotides capable of hybridizing to each strand of the first and the second primer-binding regions. The enhancer oligonucleotides may be a mixture of four oligonucleotides, each capable of hybridizing to one of the Watson strand or the Crick strand of the first or the second primer-binding regions. The enhancer oligonucleotides may also be a mixture of more than four oligonucleotides that can be grouped into four groups, each group of oligonucleotides capable of hybridizing to one of the Watson strand or the Crick strand of the first or the second primer-binding regions.

In some embodiments, at least some nucleic acids in the composition are double-stranded. In some embodiments, all nucleic acids in the composition, including target and non-target nucleic acids, probes and enhancer oligonucleotides are single-stranded.

In some embodiments, the invention is a composition for nucleic acid target enrichment comprising two or more probe oligonucleotides, each probe oligonucleotide comprising a target-binding region capable of hybridizing to a nucleic acid to be enriched, and a first and a second primer-binding region, and one or more enhancer oligonucleotides capable of hybridizing to at least one of the primer binding regions. The probe oligonucleotides in the composition are capable of specifically hybridizing under hybridization conditions, to a plurality of nucleic acid targets to be enriched present in a mixture with non-target nucleic acids. In some embodiments, the composition further comprises a mixture of target and non-target nucleic acids. In some embodiments, the mixture of target and non-target nucleic acids present in the composition is genomic DNA of an organism. In some embodiments, the mixture of target and non-target nucleic acids present in the composition is a genomic DNA library derived from the genome of an organism.

In some embodiments, hybridization between sample nucleic acids and capture probes occurs in solution. In other embodiments, hybridization occurs on solid support, e.g., surface or a slide or a particle such as bead. In this embodiment, hybridization probes are covalently or non-covalently tethered to the solid support. In some embodiments, the probes are attached to solid support via a capture moiety (e.g., biotin) present in the probes. In some embodiments, the probes are attached to solid support via hybridization of a sequence in the probe to a capture oligonucleotide covalently or non-covalently attached to solid support. The sample nucleic acids are present in solution, which is in contact with the solid support. In some embodiments, the probes are attached to solid support via primer binding sites. In such a case, the enhancer oligonucleotides of the instant invention may be used to elute the probes or probe target-complexes from the solid support.

In other embodiments, sample nucleic acids (i.e., library nucleic acids) are covalently or non-covalently tethered to the solid support (e.g., via a capture moiety present in the adaptors or another part of the library molecule) and probes are present in solution in contact with the solid support.

In some embodiments, the invention is a reaction mixture comprising a plurality of nucleic acids including target and non-target nucleic acids, two or more probe oligonucleotides, each probe oligonucleotide comprising a target-binding region, and a first and a second primer-binding region, and one or more enhancer oligonucleotides capable of hybridizing to at least one of the primer binding regions. In some embodiments, the reaction mixture incudes a plurality of probe oligonucleotides capable of specifically hybridizing under hybridization conditions, to a plurality of nucleic acid targets present in a mixture with non-target nucleic acids. In some embodiments, the reaction mixture contains genomic DNA of an organism or a genomic library from and organism. In some embodiments, all nucleic acids in the reaction mixture are single-stranded. In some embodiments, all nucleic acids in the reaction mixture are double-stranded. In some embodiments, there are four primer-binding regions on each probe and enhancer oligonucleotides bind to all four primer-binding regions. In some embodiments, the enhancer oligonucleotides comprise a mixture of four oligonucleotides, each capable of hybridizing to one of the Watson strand or the Crick strand of the first or the second primer-binding regions, or enhancer oligonucleotides comprise a mixture of more than four oligonucleotides that can be grouped into four groups, each group of oligonucleotides capable of hybridizing to one of the Watson strand or the Crick strand of the first or the second primer-binding regions. In some embodiments, the reaction mixture contains genomic DNA of an organism. In some embodiments, the reaction mixture contains a genomic library formed from genomic DNA of an organism.

In some embodiments, the invention is a kit including components and tools for performing target capture by hybridization in the presence of enhancer oligonucleotide. In some embodiments, the kit comprises an aliquot of one or more hybridization probe (each in a separate vial or as one or more probe pools) and an aliquot of one or more enhancer oligonucleotide (each in a separate vial or as a mixture of two or more enhancer oligonucleotides). In some embodiments, the kit further comprises solutions and buffers for performing hybridization and one or more post-hybridization washes. In some embodiments, the kit further comprises reagents for intermediate purification of nucleic acids, the reagents including capture particles (e.g., magnetic or paramagnetic particles) wash buffers and magnets.

In some embodiments, the kit further comprises reagents and tools for performing steps upstream of target capture by hybridization. In some embodiments, the kit comprises reagents from predating a library from nucleic acids in a sample. The library preparation reagents include one or more of DNA ligase, DNA polymerase, adaptors and buffers necessary for A-tailing and ligation of adaptors to sample nucleic acids.

In some embodiments, the kit further comprises reagents and tools for performing steps downstream of target capture by hybridization. In some embodiments, the kit comprises reagents for separation, amplification and sequencing of the captured nucleic acids.

In some embodiments, the method further comprises assessment of a disease or condition of a subject (e.g., a patient) based on the mutation status of one or more genetic loci in the patient's genome.

The mutation status is selected from no mutation (wild-type sequence), and one or more mutations selected from mutation types including at least one single nucleotide variation (SNV), at least one copy number variation (CNV), (including deletion, duplication or higher order amplification of a sequence), translocation or fusion.

In some embodiments, the invention is a method comprising enriching the patient's nucleic acids by the method described herein; determining in the enriched nucleic acids the mutation status of one or more genetic loci known to be biomarkers disease or condition, thereby detecting or diagnosing the disease or condition in the patient. In some embodiments, the method further comprises selecting or changing a treatment based on the mutation status of one or more genetic loci enriched from the patient's sample.

In some embodiments, the invention is a method of diagnosis or screening for the presence of a cancerous tumor in a patient or subject. In some embodiments, the invention includes enriching the patient's nucleic acids by the method described herein; determining in the enriched nucleic acids the mutation status of one or more genetic loci known to indicate the presence of a cancerous tumor, thereby detecting the presence of the cancerous tumor in the patient. In some embodiments, the method further comprises selecting or changing a treatment targeting the cancerous tumor based on the mutation status of one or more genetic loci enriched from the patient's sample by the method described herein.

In some embodiments, the invention is a method of monitoring the growth or shrinkage of a tumor, the method comprising periodically sampling circulating cell-free DNA (cfDNA) from a patient, enriching for one or more target sequences in the cfDNA and measuring changes in the amount cfDNA containing one or more mutation types in the target sequences, wherein an increase in the level of such mutated cell-free DNA indicates tumor growth, while a decrease in the level of such mutated cell-free DNA indicates tumor shrinkage.

In some embodiments, the invention is a method of monitoring the effectiveness of treatment of cancer in a patient or subject, the method comprising periodically sampling circulating cell-free DNA (cfDNA) from a patient, enriching for one or more target sequences in the cfDNA and measuring changes in the amount cfDNA containing one or more mutation types in the target sequences, wherein an increase in the level of such mutant cell-free DNA indicates tumor growth and ineffectiveness of treatment, while a decrease in the level of such mutant cell-free DNA indicates tumor shrinkage and effectiveness of treatment, and a stable level of such mutant cell-free DNA indicates stable disease and effectiveness of treatment.

In some embodiments, the invention is a method of diagnosis or minimal residual disease (MRD) in a cancer patient following a treatment. National Cancer Institute defines MRD as a very small number of cancer cells that remain in the body during or after treatment when the patient has no signs or symptoms of the disease. In some embodiments, the invention is a method of diagnosing MRD, the method comprising obtaining circulating cell-free DNA (cfDNA) from a patient, enriching for one or more target sequences in the cfDNA and detecting in the enriched cfDNA one or more mutation types characteristic of the tumor, wherein the presence of such mutant cell-free DNA indicates the presence of MRD in the patient.

### EXAMPLES

### Example 1. Enhancer Oligonucleotides in Target Capture

In this experiment, the probe hybridization step of the KAPA HyperCap Workflow (v3.0, available from Roche Sequencing Solutions, Inc. Pleasanton, Cal.) was performed in the presence of hybridization enhancer oligonucleotides.

To prepare for hybridization, 130 μL of KAPA HyperPure Beads were added to each tube containing the DNA Sample Library (comprised of sheared human genomic DNA ligated to adaptors) and COT Human DNA mixture. The mixture was mixed thoroughly by vortexing for 10 seconds and centrifuged. The mixture was incubated at room temperature for 10 minutes to ensure that the DNA Sample Library and COT Human DNA bind to the beads. The sample was placed on a magnet to collect the beads until the liquid was clear. The supernatant was removed and discarded. Keeping the sample on the magnet, we added 200 μL of freshly-prepared 80% ethanol and incubated the sample at room temperature for ≥30 seconds. Ethanol was removed and discarded without disturbing the beads. Residual ethanol was allowed to evaporate at room temperature.

The Hybridization Master Mix was prepared as follows:

| | |
|---|---|
| Hybridization Buffer | 28 μL |
| Hybridization Component H | 12 μL |
| PCR Grade water | 3 μL |
| Total | 43 μL |

Next, 43 μL of the Hybridization Master Mix was added to the bead-bound DNA mixture resuspended in a solution containing blocker oligonucleotides designed to bind to adaptors attached to library molecules. The reaction mixture was mixed thoroughly, centrifuged and incubated at room temperature for 2 minutes. The sample was placed on the magnet to collect the beads and incubated until the liquid was clear. We then transferred 56.4 μL of the eluate (entire volume) into a new tube containing 4 μL of the KAPA Target Enrichment Probes (a pool of biotinylated 120-nt probes) and the enhancer oligonucleotides of the instant invention. The enhancer oligonucleotides were added at four different concentrations relative to the final volume of the hybridization mixture: 0.234 mM, 0.0234 mM, 0.00234 mM and 0.000234 mM. The control reaction contained no enhancer oligonucleotides. (FIGS. 2A and 2B).

The reaction mixture was mixed thoroughly by vortexing for 10 seconds and centrifuged. Hybridization was performed in a thermocycler using the following program with the lid temperature set to 105° C., 95° C. for 5 minutes, 55° C. overnight.

The hybridized DNA was washed, recovered and amplified according to the manufacturer's recommendations of the KAPA HyperCap Workflow v3.0. The amplified DNA was sequenced on an Illumina instrument.

Results of the sequencing are shown in FIGS. 2A and 2B. FIG. 2A: dsDNA Probe Enhancer Oligonucleotides of the instant invention improved capture uniformity in a dose-dependent manner. Fold 80 Base Penalty is defined as fold additional sequencing required to bring 80% of the bases to the mean depth of coverage, therefore lower Fold 80 Base Penalty indicates better capture uniformity. FIG. 2B: inclusion of the dsDNA Probe Enhancer Oligo resulted in lower total duplicate rate in sequencing data, in a dose-dependent manner.

1-17. (canceled)

18. A composition for nucleic acid hybridization, wherein the composition comprises the following:
(a) two or more probe oligonucleotides, wherein each probe oligonucleotide comprises a target-binding region, and a first primer-binding region and a second primer-binding region; and
(b) one or more enhancer oligonucleotides, wherein the enhancer oligonucleotides are capable of hybridizing to at least one of the primer-binding regions.

19. The composition of claim 18, wherein the two or more probe oligonucleotides comprises a plurality of probe oligonucleotides capable of specifically hybridizing to a plurality of nucleic acid targets under hybridization conditions.

20. The method of claim 19, wherein hybridization conditions are stringent hybridization conditions.

21. The composition of claim 18, wherein the probe oligonucleotides are double-stranded.

22. The composition of claim 18, wherein the probe oligonucleotides are single-stranded.

23. The composition of claim 18, wherein all the probe oligonucleotides have the same first primer-binding region and the same second primer-binding region.

24. The composition of claim 18, wherein the enhancer oligonucleotides comprise a mixture of oligonucleotides capable of hybridizing to the first primer-binding region and to the second primer-binding region.

25. The composition of claim 24, wherein the enhancer oligonucleotides comprise a mixture of oligonucleotides capable of hybridizing to each strand of the first primer-binding region and the second primer-binding region.

26. The composition of claim 24, wherein the enhancer oligonucleotides comprise a mixture of four oligonucleotides, each capable of hybridizing to one of the Watson strand or the Crick strand of the first primer-binding regions or the second primer-binding regions.

27. The composition of claim 24, wherein the enhancer oligonucleotides comprise a mixture of more than four oligonucleotides that are grouped into four groups, wherein the oligonucleotides in each group is capable of hybridizing to one of the Watson strand or the Crick strand of the first primer-binding region or the second primer-binding region.

28. A composition of nucleic acid target enrichment, wherein the composition comprises the following:
(a) two or more probe oligonucleotides, wherein each probe oligonucleotide comprises a target-binding region, and a first primer-binding region and a second primer-binding region; and
(b) one or more enhancer oligonucleotides capable of hybridizing to at least one of the primer-binding regions.

29. The composition of claim 28, wherein the two or more probe oligonucleotides comprise a plurality of probe oligonucleotides capable of specifically hybridizing to a plurality of nucleic acid targets present in a mixture with non-target nucleic acids, under hybridization conditions.

30. The composition of claim 28, further comprising a mixture of target nucleic acids and non-target nucleic acids.

31. The composition of claim 29, wherein hybridization conditions are stringent hybridization conditions.

32. The composition of claim 28, wherein the probe oligonucleotides are double-stranded.

33. The composition of claim 28, wherein the probe oligonucleotides are single-stranded.

34. The composition of claim 28, wherein all the probe oligonucleotides have the same first primer-binding region and the same second primer-binding regions.

35. The composition of claim 28, wherein the one or more enhancer oligonucleotides comprise a mixture of oligonucleotides capable of hybridizing to the first primer-binding region and the second primer-binding region.

36. The composition of claim 35, wherein the enhancer oligonucleotides comprise a mixture of oligonucleotides capable of hybridizing to each strand of the first primer-binding region and the second primer-binding region.

37. The composition of claim 35, wherein the enhancer oligonucleotides comprise a mixture of four oligonucleotides, each capable of hybridizing to one of the Watson strand or the Crick strand of the first primer-binding region or the second primer-binding region.

38. The composition of claim 35, wherein the enhancer oligonucleotides comprise a mixture of more than four oligonucleotides that can be grouped into four groups, wherein the oligonucleotides in each group is capable of hybridizing to one of the Watson strand or the Crick strand of the first primer-binding region or the second primer-binding region.

39. A method of enriching for target nucleic acids, wherein the method comprises the following steps:
(a) contacting a mixture of target nucleic acids and non-target nucleic acids with a composition, wherein the composition comprises: (i) two or more probe oligonucleotides, wherein each probe oligonucleotide comprises a target-binding region, and a first primer-binding region and a second primer-binding region, and (ii) one or more enhancer oligonucleotides, wherein the enhancer oligonucleotides hybridizes to at least one of the primer binding regions;
(b) incubating the mixture of target nucleic acids and non-target nucleic acids and the composition of step (a) under hybridization conditions, such that probe oligonucleotides hybridize to target nucleic acids; and
(c) separating probe oligonucleotide-bound target nucleic acids from unbound nucleic acids.

40. The method of claim 39, wherein, in step (a), each of the target nucleic acids, the non-target nucleic acids, the two or more probe oligonucleotides, and the one or more enhancer oligonucleotides is single-stranded.

41. The method of claim 39, further comprising a step of incubating the mixture under conditions that effect denaturation of nucleic acids, prior to step (b).

42. The method of claim 39, wherein the mixture of target and non-target nucleic acids constitutes genomic DNA of an organism.

43. The method of claim 39, wherein the mixture of target nucleic acids and non-target nucleic acids constitutes a library formed from genomic DNA of an organism.

44. The method of claim 43, wherein the library formed from genomic DNA of an organism comprises one or more nucleic acids isolated from the organism, wherein the one or more nucleic acids isolated from the organism is conjugated to at least one adaptor.

45. The method of claim 44, wherein each single nucleic acid in the library is conjugated to two adaptors.

46. The method of claim 43, wherein the adaptor comprises a nucleic acid barcode.

47. The method of claim 43, wherein the adaptor comprises universal primer-binding sites.

48. The method of claim 39, further comprising a step of removing any single-stranded nucleic acids from the mixture.

49. The method of claim 48, wherein the step of removing any single-stranded nucleic acids from the mixture comprises capturing hybridized nucleic acid via a capture moiety present in the probe oligonucleotides.

50. A method of sequencing nucleic acids, wherein the method comprises the following steps:
(a) contacting a mixture of target nucleic acids and non-target nucleic acids with a composition, wherein the composition comprises: (i) two or more probe oligonucleotides, wherein each probe oligonucleotide comprises a target-binding region, and a first primer-binding region and a second primer-binding region, and (ii) one or more enhancer oligonucleotides, wherein the enhancer oligonucleotides hybridize to at least one of the primer-binding regions;
(b) incubating the mixture of target nucleic acids and non-target nucleic acids and composition of step (a) under hybridization conditions, such that probe oligonucleotides hybridize to the target nucleic acids;
(c) capturing hybrids, wherein the hybrids are formed between the probe oligonucleotides and the target nucleic acids, thereby obtaining enriched nucleic acids; and
(d) sequencing the enriched nucleic acids.

51. The method of claim 50, wherein, in step (a), each of the target nucleic acids, the non-target nucleic acids, the two or more probe oligonucleotides, and the one or more enhancer oligonucleotides is single-stranded.

52. The method of claim 50, further comprising a step of incubating the mixture under conditions that effect denaturation of nucleic acids, prior to step (b).

53. The method of claim 50, wherein the mixture of target nucleic acids and non-target nucleic acids constitutes genomic DNA of an organism.

54. The method of claim 50, wherein the mixture of target nucleic acids and non-target nucleic acids constitutes a library formed from genomic DNA of an organism.

55. The method of claim 54, wherein the library formed from genomic DNA of an organism comprises one or more nucleic acids isolated from the organism, wherein the one or more nucleic acids isolated from the organism is conjugated to at least one adaptor.

56. The method of claim 55, wherein each single nucleic acid in the library is conjugated to the two adaptor.

57. The method of claim 55, wherein the adaptor comprises a nucleic acid barcode.

58. The method of claim 55, wherein the adaptor comprises universal primer-binding sites.

59. The method of claim 50, further comprising a step of amplifying the enriched nucleic acids, prior to step (d).

60. The method of claim 59, wherein the amplifying step comprises universal primers binding to universal primer-binding sites in the enriched nucleic acids.

61. An enriched library of nucleic acids generated by the method of claim 54.

62. A reaction mixture comprising:
(a) a plurality of nucleic acids, wherein the plurality of nucleic acids comprises target nucleic acids and non-target nucleic acids;
(b) two or more probe oligonucleotides, wherein each probe oligonucleotide comprise a target-binding region, and a first primer-binding region and a second primer-binding region; and
(c) one or more enhancer oligonucleotides capable of hybridizing to at least one of the primer binding regions.

63. The reaction mixture of claim 62, wherein the two or more probe oligonucleotides comprise a plurality of probe oligonucleotides capable of specifically hybridizing to a plurality of target nucleic acids, under hybridization conditions.

64. The reaction mixture of claim 62, wherein the plurality of target nucleic acids are present in a genomic DNA of an organism.

65. The reaction mixture of claim 64, wherein hybridization conditions are stringent hybridization conditions.

66. The reaction mixture of claim 62, wherein the probe oligonucleotides are double-stranded.

67. The reaction mixture of claim 62, wherein the probe oligonucleotides are single-stranded

68. The reaction mixture of claim 62, wherein all the probe oligonucleotides have the same first primer-binding region and the same second primer-binding region.

69. The reaction mixture of claim 62, wherein the enhancer oligonucleotides comprise a mixture of oligonucleotides capable of hybridizing to the first primer-binding region and the second primer-binding region.

70. The reaction mixture of claim 69, wherein the enhancer oligonucleotides comprise a mixture of oligonucleotides capable of hybridizing to each strand of the first primer-binding region and the second primer-binding region.

71. The reaction mixture of claim 69, wherein the enhancer oligonucleotides comprise a mixture of four oligonucleotides, each capable of hybridizing to one of the Watson strand or the Crick strand of the first primer-binding region or the second primer-binding region.

72. The reaction mixture of claim 69, wherein the enhancer oligonucleotides comprise a mixture of more than four oligonucleotides that can be grouped into four groups, wherein the oligonucleotides in each group is capable of hybridizing to one of the Watson strand or the Crick strand of the first primer-binding region or the second primer-binding region.

73. The reaction mixture of claim 62, wherein the plurality of nucleic acids comprising target nucleic acids and non-target nucleic acids constitutes genomic DNA of an organism.

74. The reaction mixture of claim 62, wherein the plurality of nucleic acids comprising target nucleic acids and non-target nucleic acids constitutes a library formed from genomic DNA of an organism.

75. The reaction mixture of claim 74, wherein the library comprises nucleic acids isolated from the organism, wherein the nucleic acids are conjugated to at least adaptors.

76. The reaction mixture of claim 75, wherein each nucleic acid in the library is conjugated to two adaptor.

77. The reaction mixture of claim 75, wherein the adaptor comprises a nucleic acid barcode.

78. The reaction mixture of claim 75, wherein the adaptor comprises universal primer-binding sites.

79. The reaction mixture of claim 62, wherein each oligonucleotide probe comprises a capture moiety.

80. A method of assessment of a disease or condition in a patient, wherein the method comprises the following steps:
(a) providing a nucleic acid-containing sample from a patient;
(b) enriching target nucleic acids from the sample by the method of claim 39; and
(c) determining, in the enriched target nucleic acids, a mutation status of one or more genetic loci known to be biomarkers of the disease or condition, thereby detecting the disease or condition in the patient.

81. A method of selecting a treatment, a disease, or a condition in a patient, wherein the method comprises the following steps:
(a) providing a nucleic acid-containing sample from a patient having a disease or condition;
(b) enriching target nucleic acids from the sample by the method of claim 39; and
(c) determining, in the enriched target nucleic acids, a mutation status of one or more genetic loci known to be biomarkers of the disease or condition.

82. A method of detecting the presence of a cancerous tumor in a patient, wherein the method comprises the following steps:
(a) providing a nucleic acid-containing sample from a patient;
(b) enriching target nucleic acids from the sample by the method of claim 39; and
(c) determining, in the enriched nucleic acids, a mutation status of one or more genetic loci known to indicate the presence of a cancerous tumor, thereby detecting the presence of the cancerous tumor in the patient.

83. A method of selecting a treatment targeting a cancerous tumor in a patient based on the mutation status of the tumor, wherein the method comprises the following steps:
(a) providing a nucleic acid-containing sample from a patient;
(b) enriching target nucleic acids from the sample by the method of claim 39;
(c) determining, in the enriched nucleic acids, a mutation status of one or more genetic loci known to be mutated in a cancerous tumor; and
(d) selecting a treatment targeting the mutation status determined in step (c).

84. A method of monitoring the growth or shrinkage of a tumor, wherein the method comprises the following steps:
(a) periodically sampling circulating cell-free DNA (cfDNA) from a patient;
(b) enriching for one or more target sequences from the cfDNA by the method of claim 39; and
(c) detecting changes in the level of mutated cfDNA containing one or more mutations in the target sequences known to mutated in a cancerous tumor, wherein an increase in the level of the mutated cfDNA indicates tumor growth, while a decrease in the level of the mutated cfDNA indicates tumor shrinkage.

85. A method of monitoring the effectiveness of treatment of cancer in a patient, wherein the method comprises the following steps:
(a) periodically sampling circulating cell-free DNA (cfDNA) from a patient;
(b) enriching for one or more target sequences from the cfDNA by the method of claim 39; and
(c) detecting changes in the level of cfDNA containing one or more mutations in the target sequences known to mutated in a cancerous tumor, wherein an increase in the level of the mutant cfDNA indicates tumor growth and ineffectiveness of treatment, while a decrease in the level of the mutant cfDNA indicates tumor shrinkage and effectiveness of treatment, and a stable level of such mutant cfDNA indicates stable disease and stable effectiveness of treatment.

86. A method of detecting minimal residual disease (MRD) in a cancer patient, wherein the method comprises the following steps:
(a) obtaining circulating cell-free DNA (cfDNA) from a patient;
(b) enriching for one or more target sequences from the cfDNA by the method of claim 39; and
(c) detecting, in the enriched cfDNA, a mutation status of one or more genetic loci known to be mutated in a cancerous tumor, wherein the presence of the mutated cfDNA indicates the presence of MRD in the patient.

87. A kit for improved hybridization of nucleic acids, wherein the kit comprises the following:
(a) one or more probe oligonucleotides, each probe oligonucleotide comprises a target-binding region, and a first primer-binding region and a second primer-binding region; and
(b) one or more enhancer oligonucleotides, wherein each enhancer oligonucleotide is capable of hybridizing to the first primer-binding region and/or the second primer-binding region.

88. The kit of claim 87, wherein each probe oligonucleotide is double-stranded, and wherein the one or more enhancer oligonucleotides comprises four enhancer oligonucleotides capable of hybridizing to four primer-binding regions.

89. The kit of claim 87, further comprising reagents for purification and separation of nucleic acids.

90. The kit of claim 87, further comprising reagents for forming a library of nucleic acids.

91. The kit of claim 87, further comprising reagents for amplifying nucleic acids.

92. The kit of claim 87, further comprising reagents for sequencing nucleic acids.

93. A method of enriching for target nucleic acids, wherein the method comprises the following steps:
(a) contacting a mixture of target nucleic acids and non-target nucleic acids with a composition, thereby forming a combination of the mixture and the composition, wherein the composition comprises:
(i) two or more probe oligonucleotides, wherein each probe oligonucleotide comprises a target-binding region, and a first second primer-binding region and a second primer-binding region, wherein the first primer-binding region is hybridized to a capture oligonucleotide, wherein the capture oligonucleotide is attached to a solid support; and
(ii) one or more enhancer oligonucleotides, wherein each enhancer oligonucleotide hybridizes to the second primer-binding region;
(b) incubating the combination of the mixture and the composition under hybridization conditions, wherein the one or more enhancer oligonucleotides hybridizes to the first primer-binding region of the probe oligonucleotide; and
(c) separating probe-bound target nucleic acids from unbound nucleic acids by incubating the combination of the mixture and the composition under conditions suitable for dissociation of the first primer-binding region from the capture oligonucleotide and suitable for hybridization of the enhancer oligonucleotides to the first primer-binding region, thereby separating probe-bound target nucleic acids from unbound nucleic acids.
